(19) United States
(12) Patent Application Publication
(10) Pub. No.: US 2012/0059409 A1
(43) Pub. Date: Mar. 8, 2012

(54) Retractable Ratchet Mechanism for Surgical Instruments

(75) Inventors: Arlen J. Reschke, Longmont, CO (US); Mark J. Huseman, Broomfield, CO (US); Paul R. Romero, Loveland, CO (US); William Robinson, Aurora, CO (US)

(73) Assignee: TYCO Healthcare Group LP

(21) Appl. No.: 12/877,199

(22) Filed: Sep. 8, 2010

(51) Int. Cl. A61B 17/28

(52) U.S. Cl. 606/208

(57) ABSTRACT

A surgical instrument includes first and second shaft members each having a jaw member disposed at a distal end thereof. One (or both) of the shaft members is moveable with respect to the other from a spaced-apart position to an approximated position for grasping tissue between the jaw members. A first ratchet component is coupled to the first shaft member and defines an engagement plane. A second ratchet component is coupled to the second shaft member and is moveable between a retracted position, wherein the second ratchet component is displaced from the engagement plane, and a deployed position, wherein the second ratchet component is aligned with the engagement plane. When the second ratchet component is in the deployed position, the ratchet components are engageable with one another upon movement of the shaft member(s) to the approximated position to fix the position of the jaw members relative to one another.

### BACKGROUND

The present disclosure relates to surgical instrument and, more particularly, to a retractable ratchet mechanism for use with surgical forceps and other similar surgical instruments.

### TECHNICAL FIELD

A forceps is a plier-like instrument which relies on mechanical action between its jaws to grasp, clamp and constrict vessels or tissue. Electrosurgical forceps utilize both mechanical clamping action and electrical energy to affect hemostasis by heating tissue and blood vessels to coagulate and/or cauterize tissue. Certain surgical procedures require more than simply cauterizing tissue and rely on the unique combination of clamping pressure, precise electrosurgical energy control and gap distance (i.e., distance between opposing jaw members when closed about tissue) to “seal” tissue, vessels and certain vascular bundles.

In order to better control the clamping pressure, or closure force of the jaw members, ratchet mechanisms have been provided for locking, or fixing the relative position of the jaw members with respect to one another. These ratchet mechanisms are advantageous in that they allow the user to accurately and consistently apply a pre-determined closure force to the jaw members when grasping tissue therebetween.

In other surgical procedures, however, it may be desirable to allow the jaw members to move freely between the open and closed positions. For example, procedures wherein a precise closure force is not required and/or procedures involving rapid tissue sealing/cutting or a significant amount of tissue sealing/cutting may be facilitated by the use of a forceps capable of freely moving between the open and closed positions.

### SUMMARY

In accordance with the present disclosure, a surgical instrument is provided. The surgical instrument includes first and second shaft members each having a jaw member disposed at a distal end thereof. One (or both) of the shaft members is moveable with respect to the other from a spaced-apart position to an approximated position for grasping tissue between the jaw members. A first ratchet component is coupled to the first shaft member and defines an engagement plane. A second ratchet component is coupled to the second shaft member and is moveable between a retracted position and a deployed position. In the retracted position, the second ratchet component is displaced from the engagement plane. In the deployed position, the second ratchet component is aligned with the engagement plane. When the second ratchet component is in the deployed position, the first and second ratchet components are engageable with one another upon movement of the shaft member(s) to the approximated position to fix the position of the jaw members relative to one another.

In one embodiment, when the second ratchet component is disposed in the retracted position, the first and second shaft members are continuously moveable between the spaced-apart position and the approximated position. On the other hand, when the second ratchet component is disposed in the deployed position, the first and second shaft members are incrementally moveable from the spaced-apart position to the approximated position.

In another embodiment, the second ratchet component is pivotably coupled to the second shaft member such that the second ratchet component is rotatable between the retracted and deployed positions.

In yet another embodiment, the surgical instrument further includes a deployment mechanism coupled to the second ratchet component. The deployment mechanism is configured to move the second ratchet component between the retracted and deployed positions.

In still yet another embodiment, a locking mechanism is provided to lock the second ratchet component in the retracted position and/or the deployed position.

In accordance with another embodiment of the present disclosure, a surgical instrument is provided. The surgical instrument includes first and second shaft members each having a jaw member disposed at a distal end thereof. One (or both) of the shaft members is moveable with respect to the other from a spaced-apart position to an approximated position for grasping tissue between the jaw members. A first ratchet component is coupled to the first shaft member and a second ratchet component is coupled to the second shaft member. The second ratchet component is moveable between a retracted position and a deployed position. When the second ratchet component is disposed in the retracted position, the first and second shaft members are continuously moveable between the spaced-apart position and the approximated position. When the second ratchet component is disposed in the deployed position, the first and second shaft members are incrementally moveable from the spaced-apart position to the approximated position.

In one embodiment, the second ratchet component is pivotably coupled to the second shaft member such that the second ratchet component is rotatable between the retracted and deployed positions.

In another embodiment, a deployment mechanism coupled to the second ratchet component is provided. The deployment mechanism is configured to move the second ratchet component between the retracted and deployed positions. Further, a locking mechanism may be provided to lock the second ratchet component in the retracted position and/or the deployed position.

### BRIEF DESCRIPTION OF THE DRAWINGS

Various embodiments of the subject instrument are described herein with reference to the drawings wherein:

FIG. 1 is a side, perspective view of a forceps in accordance with one embodiment of the present disclosure including a ratchet mechanism disposed in a retracted position;

FIG. 2 is a side, perspective view of the forceps of FIG. 1 showing the ratchet mechanism disposed in a deployed position;

FIG. 3A is a transverse, cross-sectional view of the forceps of FIG. 1 wherein first and second shaft members of the forceps are disposed in a spaced-apart position and wherein the ratchet mechanism is disposed in the retracted position;

FIG. 3B is a transverse, cross-sectional view of the forceps of FIG. 1 wherein the first and second shaft members of the forceps are disposed in an approximated position and wherein the ratchet mechanism is disposed in the retracted position;

FIG. 4A is a transverse, cross-sectional view of the forceps of FIG. 1 wherein the first and second shaft members of the forceps are disposed in the spaced-apart position and wherein the ratchet mechanism is disposed in the deployed position;

FIG. 4B is a transverse, cross-sectional view of the forceps of FIG. 1 wherein first and second shaft members of the forceps are disposed in the approximated position and wherein the ratchet mechanism is disposed in the deployed position;

FIG. 5A is a side, cross-sectional view of the second shaft member of the forceps of FIG. 1 including one embodiment of a deployment mechanism shown in the retracted position;

FIG. 5B is a side, cross-sectional view of the second shaft member of the forceps of FIG. 1 wherein the deployment mechanism of FIG. 5A is shown in the deployed position;

FIG. 6A is a side, cross-sectional view of the second shaft member of the forceps of FIG. 1 including another embodiment of a deployment mechanism shown in the retracted position;

FIG. 6B is a side, cross-sectional view of the second shaft member of the forceps of FIG. 1 wherein the deployment mechanism of FIG. 6A is shown in the deployed position;

FIG. 7A is a is a side, perspective view of a forceps in accordance with another embodiment of the present disclosure including a ratchet mechanism disposed in a distal position; and

FIG. 7B is a side, perspective view of the forceps of FIG. 7A showing the ratchet mechanism disposed in a proximal position.

### DETAILED DESCRIPTION

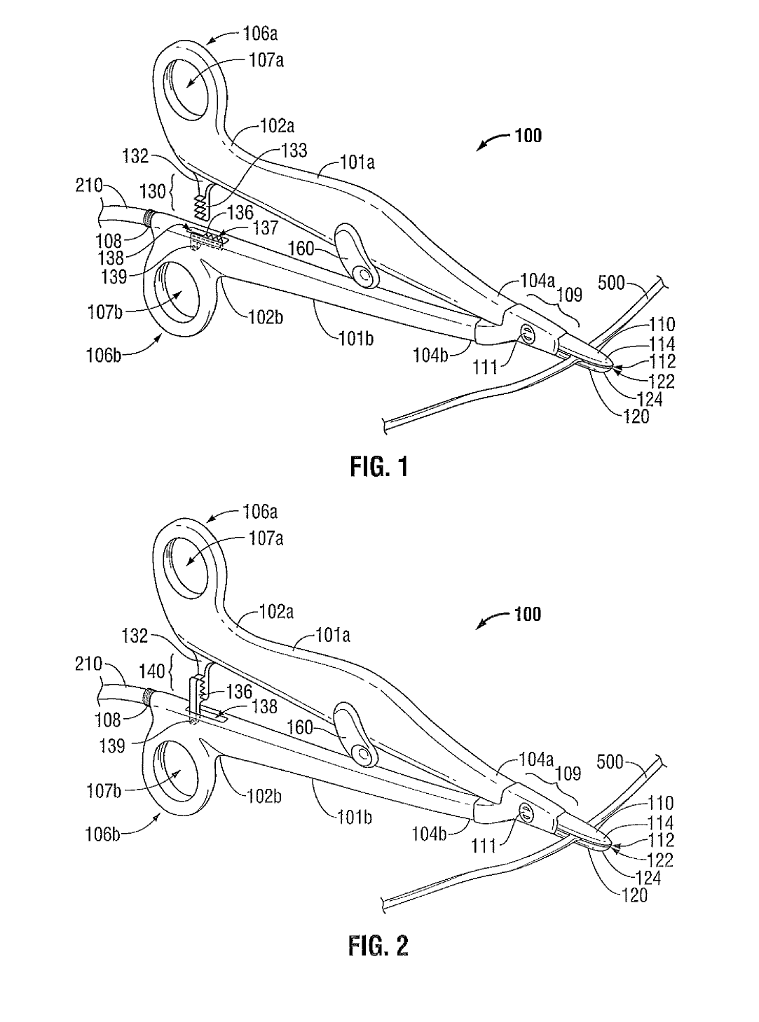

Referring initially to FIG. 1, a forceps 100 includes two elongated shaft members 101a, 101b each having a proximal end 102a, 102b and a distal end 104a, 104b, respectively. In the drawings and in the descriptions which follow, the term “proximal,” as is traditional, will refer to the end of forceps 100 (or component thereof) that is closer to the user, while the term “distal” will refer to the end that is further from the user.

Forceps 100 includes an end effector assembly 109 attached to distal ends 104a, 104b of shaft members 101a, 101b, respectively. End effector assembly 109 includes a pair of opposing jaw members 110, 120 that are pivotably connected about a pivot pin 111. Each shaft member 101a, 101b includes a handle 106a, 106b disposed at the proximal end 102a, 102b, respectively, thereof. Each handle 106a, 106b defines a finger hole 107a, 107b, respectively, therethrough for receiving a finger of the user. As can be appreciated, finger holes 107a, 107b facilitate movement of the shaft members 101a, 101b relative to one another which, in turn, pivots the jaw members 110, 120 from an open position, wherein the jaw members 110, 120 are disposed in spaced-apart relation relative to one another to a closed position, wherein the jaw members 110, 120 cooperate to grasp tissue 500 therebetween.

With continued reference to FIG. 1, one of the shafts, e.g., shaft member 101b, includes a proximal shaft connector 108 that is designed to connect the forceps 100 to a source of electrosurgical energy such as an electrosurgical generator (not shown) or other suitable power source. Proximal shaft connector 108 secures an electrosurgical cable 210 to forceps 100 such that the user may selectively apply electrosurgical energy from the generator (not shown) to either (or both) jaw member 110, 120 as needed.

As mentioned above, the two opposing jaw members 110 and 120 of end effector assembly 109 are pivotable about pivot pin 111 from the open position to the closed position for grasping tissue 500 therebetween. Jaw member 110 includes an insulated outer housing 114 that is dimensioned to mechanically engage an electrically conductive sealing surface 112 of jaw member 110. Similarly, jaw member 120 includes an insulated outer housing 124 that is dimensioned to mechanically engage an electrically conductive sealing surface 122 of jaw member 120. Electrically conductive sealing surfaces 112 and 122 are opposed to one another, such that, upon activation, electrosurgical energy may be supplied to the electrically conductive sealing surfaces 112 and 122 for sealing tissue 500 disposed between the jaw members 110 and 120. More particularly, a first electrical potential may be provided to first jaw member 110 and a second electrical potential may be provided to second jaw member 120 to conduct energy between the sealing surfaces 112, 122 of jaw members 110, 120, respectively, to seal tissue 500 disposed therebetween.

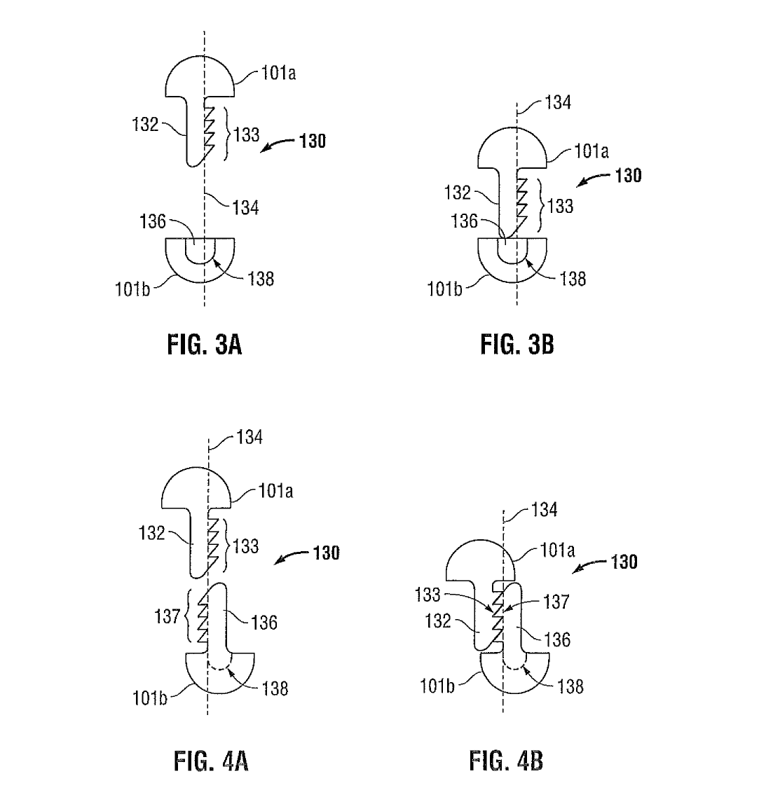

With reference now to FIGS. 1 and 2, a ratchet mechanism 130 including first and second ratchet components 132, 136 is disposed at proximal ends 102a, 102b of shaft members 101a, 101b, respectively, for selectively locking jaw members 110, 120 relative to one another at various positions during pivoting. First ratchet component 132 is engaged to first shaft member 101a and extends therefrom toward second shaft member 101b. First ratchet component 132 includes a plurality of locking teeth 133 disposed thereon for selectively engaging corresponding ratchet teeth 137 of second ratchet component 136. Locking teeth 133 of first ratchet component 132 define an engagement plane 134 (see FIGS. 3A-4B). Second ratchet component 136 is pivotably coupled to second shaft member 101b and, as mentioned above, includes a plurality of locking teeth 137 disposed thereon and configured to engage locking teeth 133 of first ratchet component 132. Second ratchet component 136 is moveable, or pivotable between a retracted position (FIG. 1) and a deployed position (FIG. 2). However, first ratchet component 132 may be moveable between a retracted position and a deployed position while second ratchet component is fixedly engaged to shaft member 101b, or, alternatively, both first and second ratchet components 132, 136, respectively, may be moveable between a retracted position and a deployed position.

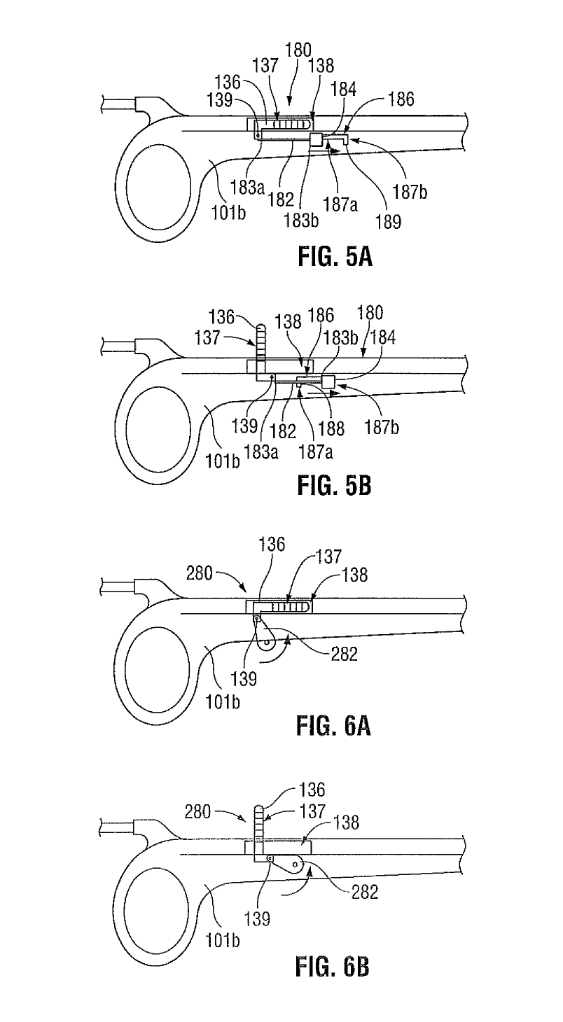

When second ratchet component 136 is disposed in the retracted position, as shown in FIG. 1, second ratchet component 136 is positioned within recessed portion 138 of shaft member 101b such that locking teeth 137 of second ratchet component 136 are positioned off of, or displaced from engagement plane 134, i.e., such that second ratchet component 136 is out of alignment with first ratchet component 132. In the deployed position, second ratchet component 136 is rotated about pivot 139 to extend from recessed portion 138 of shaft member 101b toward shaft 101a, as shown in FIG. 2, such that locking teeth 137 of second ratchet component 136 are aligned with engagement plane 134, i.e., such that first and second ratchet components 132, 136, respectively, are substantially aligned with one another. As will be described in greater detail below, a deployment mechanism, e.g., deployment mechanisms 180, 280 (FIGS. 5A-5B and 6A-6B, respectively), may be provided for moving second ratchet component 136 between the retracted and deployed positions. Deployment mechanisms 180, 280 (FIGS. 5A-5B and 6A-6B, respectively) may further include a locking feature for fixing second ratchet component 136 in the retracted position and/or the deployed position.

Turning now to FIGS. 3A and 3B, and as mentioned above, second ratchet component 136 is pivotably moveable between a retracted position (FIGS. 1 and 3A-3B) and a deployed position (FIGS. 2 and 4A-4B) for moving locking teeth 137 into alignment with engagement plane 134. With second ratchet component 136 disposed in the retracted position, i.e., when locking teeth 137 are displaced from engagement plane 134, shaft members 101a, 101b are freely moveable with respect to one another to open and close jaw members 110, 120 (FIG. 1). In other words, since second ratchet component 136 is not aligned with first ratchet component 132 when second ratchet component 136 is disposed in the retracted position, locking teeth 133 of first ratchet component 132 and locking teeth 137 of second ratchet component 136 are not engageable with one another for fixing the relative position of shaft members 101a, 101b upon movement of shaft members 101a, 101b from the spaced-apart position (FIG. 3A) to the approximated position (FIG. 3B). Thus, in the retracted position, first and second ratchet component 132, 136, respectively, do not inhibit the motion of shaft members 101a, 101b between the spaced-apart and approximated positions, i.e., shaft members 101a, 101b are continuously moveable between the spaced-apart and approximated positions when second ratchet component 136 is in the retracted position.

On the other hand, as shown in FIGS. 4A-4B, when second ratchet component 136 is moved to the deployed position, locking teeth 133 of first ratchet component 132 and locking teeth 137 of second ratchet component 136 are aligned on engagement plane 134. Upon movement of shaft members 101a, 101b from the spaced-apart position (FIG. 4A) to the approximated position (FIG. 4B) with second ratchet component 136 disposed in the deployed position, e.g., with locking teeth 133 of first ratchet component 132 and locking teeth 137 of second ratchet component 136 aligned on engagement plane 134, locking teeth 133, 137 of ratchet components 132, 136, respectively, are engaged with one another to lock, or fix the position of jaw members 110, 120. More specifically, locking teeth 133 of first ratchet component 132 and locking teeth 137 of second ratchet component 136 each define opposed saw-toothed configurations such that, as shaft members 101a, 101b are moved from the spaced-apart position (FIG. 4A) to the approximated position (FIG. 4B) to grasp tissue 500 (FIG. 1) between jaw members 110, 120 (FIG. 1), locking teeth 133 of first ratchet component 132 and locking teeth 137 of second ratchet component 136 are ramped over one another, incrementally engaging each successive tooth 133, 137 of the opposed ratchet component 132, 136. However, shaft members 101a, 101b are inhibited from returning back toward the spaced-apart position when locking teeth 133, 137 are engaged with one another due to the saw-toothed configuration of locking teeth 133, 137. Thus, shaft members 101a, 101b may be moved toward the approximated position when second ratchet component 136 is deployed such that first and second ratchet components 132, 136, respectively, engage one another to fix the relative position of shaft members 101a, 101b and jaw members 110, 120. Accordingly, the user may then release shaft member 101a and/or shaft member 101b, while ratchet mechanism 130 maintains jaw members 110, 120 in the fixed position.

As mentioned above, the plurality of locking teeth 133, 137 of first and second ratchet components 132, 136, respectively, are capable of incrementally engaging one another. Each increment of engagement of first ratchet component 132 and second ratchet component 136 corresponds to a specific position of shaft members 101a, 101b and, accordingly, to a specific closure force applied to jaw members 110, 120. Thus, with second ratchet component 136 in the deployed position, the user may accurately apply a pre-determined closure force to jaw members 110, 120 by moving shaft members 101a, 101b toward the approximated position to incrementally engage first and second ratchet components 132, 136 to a desired engagement position. For example, where a relatively small closure force is desired, first and second shaft members 101a, 101b may be moved such that ratchet components 132, 136 are only slightly overlapping and interlocking. However, where a relatively large closure force is desired, first and second shaft members 101a, 101b may be approximated further such that first and second ratchet components 132, 136 are substantially overlapping and interlocking with one another.

The number, configuration and/or spacing of locking teeth 133 of first ratchet component 132 and locking teeth 137 of second ratchet component 136 define the incremental locking positions of shaft members 101a, 101b and, thus, the set of closure pressures for jaw members 110, 120. As such, locking teeth 133, 137 may be spaced closer-together, or farther apart along ratchet components 132, 136, respectively, depending on whether finer or more coarse closure pressure increments are desired. Additionally, ratchet mechanism 130 may include a plurality of visual markings (not shown) and/or may include audible and/or tactile feedback features for confirming the incremental position of ratchet mechanism 130 and, thus, for alerting the user as to the amount of closure force supplied to jaw members 110, 120.

Referring now to FIGS. 5A-5B one embodiment of a deployment mechanism 180 for moving, or pivoting second ratchet component 136 between the retracted and deployed positions is shown. Deployment mechanism 180 is disposed on shaft member 101b and includes a linkage 182, a slide trigger 184 and a slide track 186. Linkage 182 is coupled to second ratchet component 136 at a first end 183a thereof and to slide trigger 184 at a second end 183b thereof. Slide trigger 184 is engaged within and is longitudinally translatable along slide track 186 from a proximal end 187a of slide track 186 to a distal end 187b of slide track 186. As shown in FIG. 5A, when slide-trigger 184 is disposed at proximal end 187a of slide track 186, second ratchet component 136 is disposed in the retracted position. Slide track 186 may include a proximal locking segment 188 that is recessed from slide track 186 such that slide trigger 184 may be fixed, or locked at proximal end 187a of slide track 186, thereby fixing, or locking second ratchet component 136 in the retracted position.

In order to deploy second ratchet component 136 from the retracted position to the deployed position, slide trigger 184 is first moved from proximal locking segment 188 into slide track 186 and is then translated distally along slide track 186 toward distal end 187b thereof. As slide trigger 184 is translated distally, linkage 182 is similarly pulled distally. Distal translation, or pulling of linkage 182, in turn, pulls second ratchet component 136 distally, thereby rotating second ratchet component 136 about pivot 139 to the deployed position, as shown in FIG. 5B. Distal end 187b of slide track 186 may include a distal locking segment 189, similar to proximal locking segment 188, for locking, or fixing the position of slide trigger 184 at distal end 187b of slide track 186 and, thus, for fixing second ratchet component 136 in the deployed position. To return second ratchet component 136 back to the retracted position, slide trigger 184 is disengaged from distal locking segment 189 and is translated proximally along slide track 186 such that linkage 182 urges second ratchet component 136 proximally, rotating second ratchet component 136 about pivot 139 back to the retracted position shown in FIG. 5A.

Turning now to FIGS. 6A-6B, another embodiment of a deployment mechanism 280 for moving second ratchet component 136 between the retracted and deployed positions is shown. Deployment mechanism 280 includes a rotatable actuator 282 engaged to pivot 139 of second ratchet component 136 such that rotation of rotatable actuator 282 effects rotation of second ratchet component 136. As can be appreciated, rotatable actuator 282 may be rotated between a first position, as shown in FIG. 6A, and a second position, as shown in FIG. 6B, for rotating second ratchet component 136 between the retracted position and the deployed position. Deployment mechanism 280 (and/or deployment mechanism 180) may further include a locking mechanism (not shown) for locking second ratchet component 136 in either or both of the retracted and deployed positions. Further, other deployment mechanisms for moving second ratchet component 136 between the retracted and deployed positions are also contemplated, e.g., solenoid pin mechanisms, electromagnetic mechanisms, or other suitable mechanical or electromechanical deployment mechanisms.

With reference to FIGS. 1 and 2, the use and operation of forceps 100 will be described. Initially, it is determined whether forceps 100 is to be operated in an incremental mode, corresponding to the deployed position of second ratchet component 136, or a continuous mode, corresponding to the retracted position of second ratchet component 136. This determination may be made in part based upon the particular surgical procedure to be performed. For example, where a more controlled, precise application of closure pressure to tissue 500 grasped between jaw members 110, 120 is desired, the incremental mode is selected, i.e., second ratchet member 136 is moved to the deployed position. On the other hand, where multiple tissue segments 500 are to be sealed in a more time-sensitive environment, the continuous mode may be selected, i.e., second ratchet member 136 is moved to the retracted position.

In the incremental mode, wherein second ratchet member 136 is in the deployed position, forceps 100 is positioned such that tissue 500 to be grasped and sealed is disposed between jaw members 110, 120. Shaft members 101a, 101b are then moved from the spaced-apart position toward the approximated position to move jaw members 110, 120 toward the closed position to grasp tissue 500 therebetween. Upon movement of shaft members 101a, 101b toward the approximated position, as mentioned above, locking teeth 133, 137 of first and second ratchet components 132, 136, respectively, engage one another. As shaft members 101a, 101b are further approximated, locking teeth 133, 137 ramp over one another such that first and second ratchet components 132, 136 are further engaged and further overlapped with one another. As discussed above, shaft members 101a, 101b may be approximated to the desired increment of engagement corresponding to the desired closure pressure, as confirmed by visual markings (not shown) on forceps 100 and/or audible, tactile, or other feedback to the user. Once the desired position is reached, the user no longer needs to maintain a specific closure pressure on shaft members 101a, 101b but, rather, may loosen the grasp of forceps 100 since the incremental engagement of teeth 133, 137 of first and second ratchet components 132, 136, respectively, of ratchet mechanism 130 maintains the desired closure pressure on tissue 500 grasped between jaw members 110, 120.

With tissue 500 grasped between jaw members 110, 120 at the desired closure pressure, electrosurgical energy may be supplied to jaw members 110, 120 and, more particularly, to sealing surfaces 112, 122 of jaw members 110, 120, respectively, for conduction through tissue 500 to effect a tissue seal. After tissue 500 has been sealed, a knife (not shown) may be advanced from shaft member 101a (or shaft member 101b) between jaw members 110, 120 to divide tissue 500 along the previously-formed tissue seal. First and second ratchet components 132, 136 of ratchet mechanism 130 may then be disengaged such that shaft members 101a, 101b and jaw members 110, 120 may be returned to the open, or spaced-apart position to release the sealed and divided tissue 500.

In the continuous mode, wherein second ratchet member 136 is in the retracted position, forceps 100 is initially positioned such that tissue 500 to be grasped and sealed is disposed between jaw members 110, 120. Next, shaft members 101a, 101b are moved toward the approximated position to grasp tissue between jaw members 110, 120. Since second ratchet component 136 is out of alignment with first ratchet component 132, shaft members 101a, 101b are continuously, rather than incrementally, moveable between the spaced-apart and approximated position. However, in the continuous mode, the user is required to maintain shaft members 101a, 101b in the desired position to maintain a consistent closure pressure on tissue 500 grasped between jaw members 110, 120.

With tissue grasped between jaw members 110, 120, electrosurgical energy may be supplied to sealing surfaces 112, 122 of jaw members 110, 120, for sealing tissue 500. A knife (not shown) may then be advanced through tissue 500 to divide the previous-sealed tissue 500. Once tissue 500 has been sealed and/or cut, the user may simply move shaft members 101a, 101b back to the approximated position, releasing tissue 500 from jaw members 110, 120. Forceps 100 may then be positioned adjacent the next segment of tissue 500 to be sealed and/or cut and the above-described process may be repeated. As can be appreciated, obviating the need to disengage, or release ratchet mechanism 130 after each successive grasp/seal/cut operation facilitates the sealing and/or cutting of multiple segments of tissue 500 during the procedure.

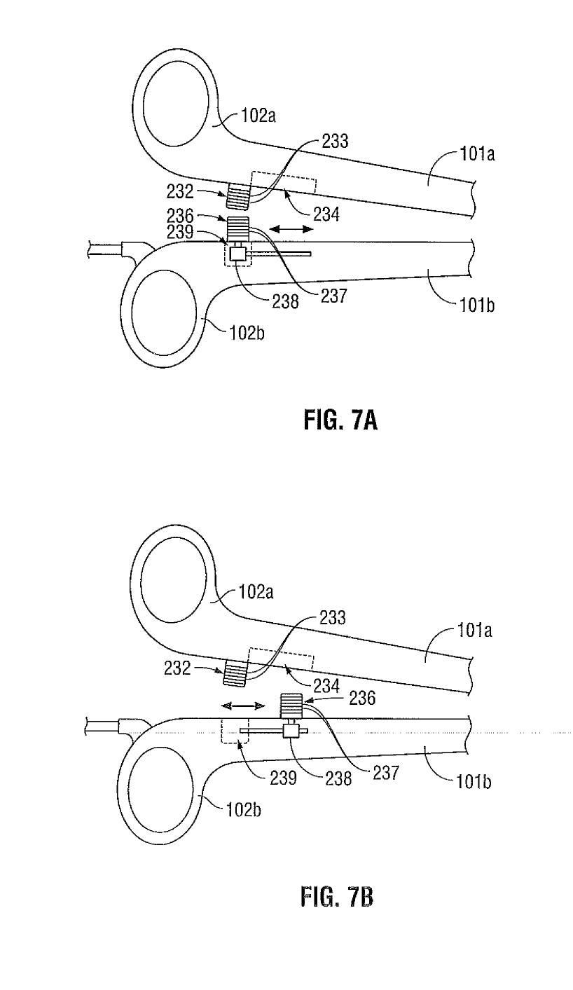

Turning now to FIGS. 7A and 7B, another embodiment of a ratchet mechanism 230 for use with forceps 100 is shown. Ratchet mechanism 230 is similar to ratchet mechanism 130 and includes first and second ratchet components 232, 236, respectively, disposed at proximal ends 102a, 102b of shaft members 101a, 101b, respectively, for selectively locking jaw members 110, 120 (FIG. 1) relative to one another at various positions during pivoting. First ratchet component 232 is coupled to first shaft member 101a and includes a plurality of locking teeth 233 disposed thereon for selectively engaging corresponding locking teeth 237 of second ratchet component 236. Second ratchet component 236 is slidably coupled to second shaft member 101b and, as mentioned above, includes locking teeth 237 configured to engage locking teeth 233 of first ratchet component 232.

As shown in FIGS. 7A-7B, second ratchet component 236 of ratchet mechanism 230 is moveable, or slidable along shaft member 101b from a proximal position (FIG. 7A) to a distal position (FIG. 7B). Alternatively, first ratchet component 232 may be moveable, or slidable between a proximal and a distal position for with respect to first shaft member 101a, or both ratchet components 232, 236 may be slidable along respective shaft members 101a, 101b. In either embodiment, a slidable tab 238 may be provided for moving first ratchet component 232 and/or second ratchet component 236 between the proximal and distal positions.

Similar to the previous embodiments, when second ratchet component 236 is disposed in the distal position (FIG. 7B), teeth 237 of second ratchet component 236 are displaced from teeth 233 of first ratchet component 232 such that shaft members 101a, 101b may be moved continuously between the spaced-apart and approximated positions, e.g., the continuous mode. Shaft members 101a, 101b may further include slots 234, 239, respectively, defined therein to accommodate ratchet components 232, 236 therein when second ratchet component 236 is disposed in the distal position, e.g., to permit full approximation of shaft members 101a, 101b. When second ratchet component 236 is moved to the proximal position (FIG. 7A), locking teeth 237 of second ratchet component 236 are moved into aligned with locking teeth 133 of first ratchet component 232, such that shaft members 101a, 101b may be moved incrementally, due to the incremental engagement of first and second ratchet components 232, 236, respectively, from the spaced-apart position to the approximated position, e.g., the incremental mode. The features and operation of ratchet mechanism 230 in conjunction with forceps 100 are otherwise similar to those described in the above embodiments.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical instrument, comprising:
first and second shaft members each having a jaw member disposed at a distal end thereof, at least one of the shaft members moveable with respect to the other from a spaced-apart position to an approximated position for grasping tissue between the jaw members;
a first ratchet component coupled to the first shaft member and defining an engagement plane; and
a second ratchet component coupled to the second shaft member and moveable between a retracted position, wherein the second ratchet component is displaced from the engagement plane, and a deployed position, wherein the second ratchet component is aligned with the engagement plane,
wherein, when the second ratchet component is in the deployed position, the first and second ratchet components are engageable with one another upon movement of the at least one shaft member to the approximated position to fix the position of the jaw members relative to one another.

2. The surgical instrument according to claim 1, wherein, when the second ratchet component is disposed in the retracted position, the first and second shaft members are continuously moveable between the spaced-apart position and the approximated position.

3. The surgical instrument according to claim 1, wherein, when the second ratchet component is disposed in the deployed position, the first and second shaft members are incrementally moveable from the spaced-apart position to the approximated position.

4. The surgical instrument according to claim 1, wherein the second ratchet component is pivotably coupled to the second shaft member such that the second ratchet component is rotatable between the retracted and deployed positions.

5. The surgical instrument according to claim 1, further comprising a deployment mechanism coupled to the second ratchet component, the deployment mechanism configured to move the second ratchet component between the retracted and deployed positions.

6. The surgical instrument according to claim 1, further comprising a locking mechanism configured to lock the second ratchet component in at least one of the retracted position and the deployed position.

7. A surgical instrument, comprising:
first and second shaft members each having a jaw member disposed at a distal end thereof, at least one of the shaft members moveable with respect to the other from a spaced-apart position to an approximated position for grasping tissue between the jaw members;
a first ratchet component coupled to the first shaft member; and
a second ratchet component coupled to the second shaft member and moveable between a retracted position and a deployed position,
wherein, when the second ratchet component is disposed in the retracted position, the first and second shaft members are continuously moveable between the spaced-apart position and the approximated position and wherein, when the second ratchet component is disposed in the deployed position, the first and second shaft members are incrementally moveable from the spaced-apart position to the approximated position.

8. The surgical instrument according to claim 7, wherein the second ratchet component is pivotably coupled to the second shaft member such that the second ratchet component is rotatable between the retracted and deployed positions.

9. The surgical instrument according to claim 7, further comprising a deployment mechanism coupled to the second ratchet component, the deployment mechanism configured to move the second ratchet component between the retracted and deployed positions.

10. The surgical instrument according to claim 7, further comprising a locking mechanism configured to lock the second ratchet component in at least one of the retracted position and the deployed position.
